(19) United States
(12) Patent Application Publication
(10) Pub. No.: US 2009/0157219 A1
(43) Pub. Date: Jun. 18, 2009

(54) Intelligent Sleeve Container for Use in a Controlled Syringe System

(76) Inventors: Lance T. Parker, JR., Houston, TX (US); Theresa W. Fossum, College Station, TX (US); Douglas Smith, Albuquerque, NM (US); Reed Killion, Spring, TX (US)

Correspondence Address: JONES, WALKER, WAECHTER, POITEVENT, CARRERE;& DENEGRE, L.L.P., 5TH FLOOR, FOUR UNITED PLAZA, 8555 UNITED PLAZA BOULEVARD, BATON ROUGE, LA, 70809, US

(21) Appl. No.: 12/114,267

(22) Filed: May 2, 2008

Related U.S. Application Data
(60) Provisional application No. 60/915,827, filed on May 3, 2007.

(51) Int. Cl. G06F 17/00; A61M 5/00

(52) U.S. Cl. 700/231; 604/48

(57) ABSTRACT

Various embodiments of the present invention concern improving the dosing accuracy and efficacy of systems that calculate medicine doses for injection in animals and prepare the correct dose in an automated syringe subsystem. In an embodiment, the improvement is gained by simultaneously protecting the medicine from ambient outdoor temperatures that thermally degrade its efficacy while precisely monitoring the amount of medicine remaining inside the source vial to prevent inadvertent under-dosing due to undetected source depletion. This dual-sleeve system (one for temperature, one for data storage concerning the source vial contents) forms an intelligent adjunct to current and future automated injection technology used to maintain healthy livestock.

### PRIORITY CLAIM

this invention claims the priority benefit of U.S. provisional application No. 60/915,827 filed on May 3, 2007, and which application is hereby incorporated by reference.

### FIELD OF THE INVENTION

The present invention relates generally to storage systems for medications for delivery and use in a field application.

### BACKGROUND INFORMATION

It is often desirable to treat large numbers of individuals or animals, referred to herein generally as subjects, with a substance, such as a medication or other material, with speed, efficiency, and accuracy. One such system is disclosed in U.S. Pat. No. 7,056,307 (the “'307 patent”), which is incorporated herein by reference in its entirety. This system discloses a highly accurate pump, reservoir bottle and syringe, where the pump delivers precise dosages of fluid medication from the reservoir bottle to the syringe for delivery. A system computer determines the dosage to be injected to a given animal (generally determined by animal weight, medical history, medication records, and other pertinent information), and instructs the pump controller (which can be a computer) to dispense the calculated amount of fluid. The pump then dispenses the proper amount of fluid (possibly in several doses, under the control of the system computer) to the syringe for injection into the animal by an operator or technician. The '307 system, or a comparable dosage controlled injection system provides for ready administration of medications in accurate amounts and time-stamping the administration of the dose.

Much of the equipment in the '307 system can be housed in a easily transportable portable carrier, such as a backpack or belt mounted holster system for use in the field, such as a feedlot in the pastures. The system computer interfaces the field deployed equipment by wireless communication from a wireless device associated with the field components. In this fashion, animal data (such as a stored weight of the animal, medical history, ownership information) and dosage information can be exchanged between the system and field deployed components. The system computer could be located in the transportable carrier if the needed animal data was stored in memory accessible by the system computer, for instance, needed animal data could be retrieved from a data storage in the system computer or from data storage located on the animal itself, such as stored in an RFID tag located on the animal. Other aspects of the controlled injection system include a sensor for monitoring the status of the syringe (U.S. provisional application No. 61/039,158, hereby incorporated by reference) and improved spool valve for filling the syringe, as disclosed in U.S. provisional application No. 61/038,351, also incorporated by reference).

For field dispensal of medications dependent on the animal's weight, a portable battery operated or mechanical scale can be incorporated in the '307 system, with the system receiving the weight either electronically or from manual operator input. In a feedlot environment, a scale is generally available and can be incorporated into the controlled injection system (i.e. communicate with the host computer, either manual input or electronic input).

At times it is desirable or necessary to mass vaccinate animals in the field or on a feedlot, such as vaccination of cattle in response to a condition or in response to a disease indicator. Multiple medications may be needed for administration in harsh environments and under confusing adverse conditions. In these situations, medications must be delivered in large volumes and exposed to ambient conditions. A system is needed to protect medication from environmental conditions, and to assist in the identification and tracking of the medications administered or remaining to be administered.

### SUMMARY OF THE INVENTION

One embodiment of the invention includes a memory containing sleeve that is associated with a medication vial or bottle. The memory sleeve stores relevant information concerning the medication and can communicate with a computer system to transmit the stored information, and, in certain embodiments, the stored information may be updated. Stored information can comprise medicine identification, security status, temperature status, volume available, date first used, or any other desired characteristic. In one embodiment the memory sleeve may be a ring-shaped, electronically active system that fits over the lip of the medication source vial and grips the narrower neck of the vial when properly seated and locked in position. This sleeve may incorporate a wireless communications device and a non-volatile memory storage device, as well as means (either direct or indirect) for determining the amount of medication being used or remaining. An appropriate socket for direct, wired communication (e.g., USB, serial, and/or the like) may optionally be included in the sleeve for communication with onboard components. The memory sleeve may include a screen for input/output purposes, such as a screen on a calculator driven by battery or solar powered, or a touch screen for direct input.

In one embodiment the invention includes an insulating jacket or sleeve for the medicine reservoir, providing thermal stability for the contained fluids as well as protection from light energy degradation. In another embodiment, the two sleeves in combination are include in the scope of the invention.

These and other features and advantages of the invention will be apparent to those skilled in the art from the following detailed description of an embodiment, taken together with the accompanying figures and claims.

### BRIEF DESCRIPTION OF THE DRAWINGS

FIG. 1 includes two notional combinations of Memory Sleeve, medication source vial, and a Temperature Sleeve, where

FIG. 1a illustrates an exploded view of an embodiment of three components taught in the present invention.

FIG. 1b illustrates an embodiment of the three components ready for deployment.

FIG. 2 illustrates variants of the Memory Sleeve component in relation to the medication source vial.

FIG. 2a illustrates a Memory Sleeve that totally encompasses the lip of the source vial.

FIG. 2b illustrates a Memory Sleeve that seals the Temperature Sleeve to improve the latter's temperature stabilization capabilities.

FIG. 3 illustrates alternate embodiments of the present invention.

FIG. 3a illustrates an alternate embodiment of the present invention.

FIG. 3b illustrates an embodiment of a locking architecture for the Memory Sleeve.

FIG. 4 illustrates a system architecture relative to the Memory Sleeve's construction.

FIG. 4a illustrates an embodiment of the present invention.

FIG. 4b illustrates an alternate embodiment of the present invention.

FIG. 4c illustrates an alternate embodiment of the present invention.

### DETAILED DESCRIPTION OF THE INVENTION

The particulars shown herein are by way of example and for purposes of illustrative discussion of the preferred embodiments of the present invention. In this regard, structural details of the invention in more detail than is necessary for the fundamental understanding of the invention have been excluded, the description taken with the drawings and/or examples making apparent to those skilled in the art how the several forms of the invention may be embodied in practice.

### Description of the Preferred Embodiment

The present invention teaches the deployment of dual-sleeve structure that addresses thermal stability and monitoring of vial fluids. Either sleeve can be used alone or in combination with the other.

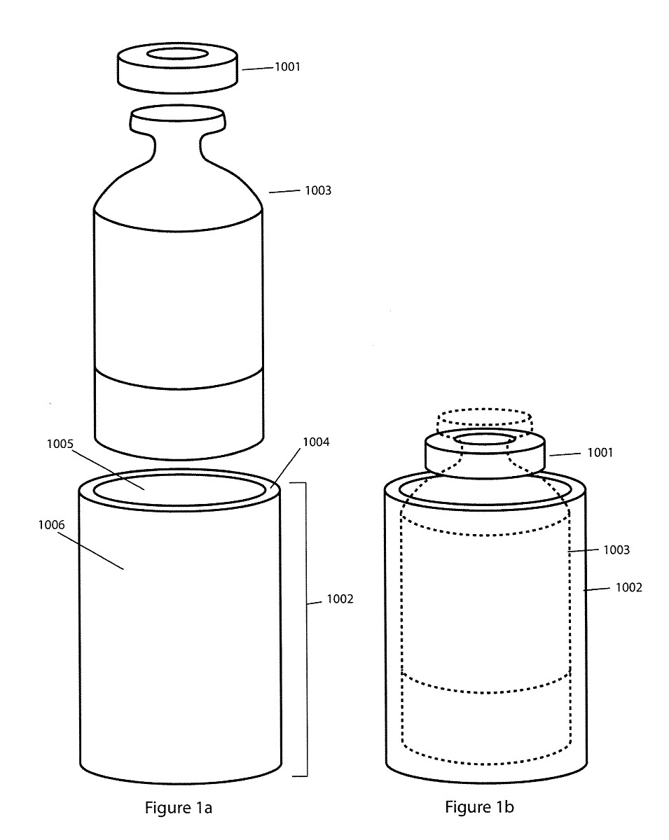

FIG. 1 illustrates the basics of the present invention. FIG. 1a shows an exploded view of the two sleeves, 1001 and 1002, in relation to the medicine source vial 1003, which is also commonly referred to as a bottle, container, vial, reservoir, ampoule, or other recognized medicine storage and delivery system. It can be seen that the source vial 1003 is shown with a conventional bottle neck and lip structure, and it can have an industry-standard protective cap and a hermetic membrane seal (such as a foil membrane seal) that can be pierced or punctured by a hollow needle, such as may be part of a hypodermic needle filling system.

### The Insulating Sleeve

The insulating sleeve 1002 is shown as a cylinder that surrounds the vial. The insulating sleeve provides temperature stabilization by providing thermal insulation within a relatively thin package. In a preferred embodiment, the present invention incorporates thermal insulation technology developed by NanoPore Incorporated of New Mexico (and assigned to NanoPore Insulation LLC) and commercially sold as NanoPore HP150 or NanoTherm vacuum insulation. A preferred application is the material in the form of a vacuum insulation panel or VIP. VIPs are made by sealing the thermal insulation in a barrier film under vacuum. The barrier film (a polymer or metal film) is formed into a pouch which the panel insert is put inside before the unit is evacuated and heat sealed. The sealed edges of the barrier film creates a flap of film which extends out from the edges of the panel which can be folded and taped against the panel in use. The VIP structure creates a three layer structure: an inside layer 1005 formed by a semi-pliable or pliable thin material, such as a polymer or metal film, a similar pliable material forming the outer layer 1006 that is opaque to radiation, including infrared radiation, to reduce radiant energy transfer from the medicine source, and an interstitial matrix 1004 of nanoporous material with ultra low thermal conductivity due to exploitation of the Knudsen Effect. It can be appreciated that the inner and outer layers which encapsulate the inner nanoporous matrix can form a contiguous, continuous surface and may thus be identical. While the Nanopore VIP is preferred due to the long term temperature stability it provides, other common insulating materials could be used for sleeve construction, particularly where long term exposure is not anticipated.

As shown in FIG. 1, the insulating sleeve covers a substantial portion of the vial exterior. As shown, a portion of the top exterior is left exposed for coupling of the memory sleeve. Heat loss through this area is reduced as liquid level, even in a full vial, should be below the top of the insulating layer. However, if needed, the insulating layer can cover the entire exterior surface of the vial. The insulating sleeve in combination with a annular memory sleeve may cover substantially all of the exterior surface of the vial as shown in FIG. 1. Alternatively, the vial itself may be manufactured from a highly insulating material, thereby combining the vial and sleeve into a single device.

### The Memory Sleeve.

The present invention embraces a memory sleeve 1001 that is to be affixed to the source vial, here shown as affixed to the upper portion of the source vial. FIG. 1b illustrates one possible form that the assembled system might take, wherein the memory sleeve 1001 is, in this example, affixed below the widened rim of the bottle neck at the distal end of the medicine source vial 1003.

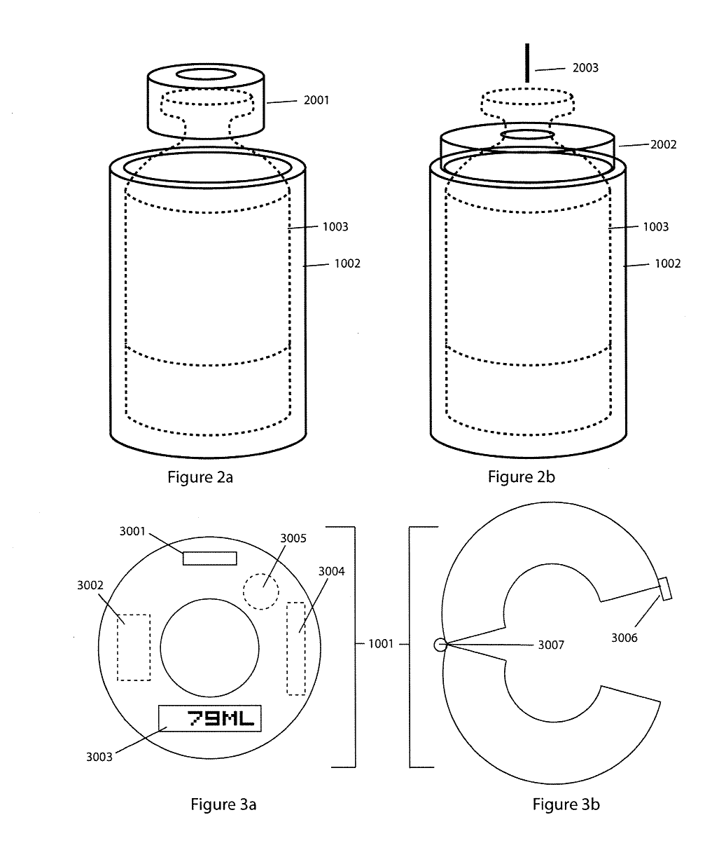

Other geometric variations are possible, for instance, the memory sleeve may be a semi-planar surface attached to the vial, such as glued to the bottom of the vial (either interior to the memory sleeve, exterior to the memory sleeve, or with a memory sleeve that lacks a bottom. FIG. 2 illustrates additional geometric variations on the design and deployment of the memory sleeve structure near the top portion of the vial, and illustrates that the sleeve need not be structured to fit around the narrowest portion of the bottle neck, but can be fabricated to lock onto the rim. In such cases, the inner diameter of the memory sleeve 2001 might be a pliable material, such as a semi-rigid polymer foam, shaped internally to provide maximum stability when forcibly placed over the source vial's rim as shown, the cavity inside the inner diameter of the annulus approximating the shape of the vial rim. The actual location of the memory sleeve is not critical.

FIG. 2b shows an alternate approach to configuration of the memory sleeve. In this example, the memory sleeve is seated around the narrowest part of the source vial 1003, but the diameter of this sleeve 2002 is increased and its relative position, in relation to the insulating sleeve 1002, is adjusted so that the memory sleeve 2001 effectively seals against the insulating sleeve 1002. FIG. 2b also illustrates part of the larger system into which the present invention is to be integrated, namely, the hollow needle 2003 that will be inserted and attached via flexible cable to the high-precision viscosity-independent pump (not shown) that pumps medication out of the source vial 1003. The hollow needle 2003 is intended to pass through the sealed membrane of the source vial to preserve sterility of the medicine, as has become standard practice in regard to medications delivered via injection for both animals and humans. A vent needle would also be inserted into the sealed membrane to prevent vacuum lock when in use. The two needles may be combined into a single needle structure.

Whereas the annular body of the memory sleeve 1001 (which may be fabricated from plastics, polymers, composites, or any environmentally suitable material in which the active components being herein described may be mounted and, if necessary, sealed from the outside world) is depicted as an undifferentiated solid, it is understood that it can be formed with cavities and mounting hardware and/or fastener anchor points as well as wiring or cabling between different possible components, as needed. The memory device 3002 is shown inhabiting a suitably formed cavity inside the annulus into which the device is positioned so as to be able to withstand shock and vibration to the limits specified for the medication dispensing system.

FIG. 3 provides details concerning the memory sleeve and potential component(s) within it that provides its intended functionality as an intelligent device. FIG. 3a shows a representative plan view of one possible configuration of components comprising an intelligent memory sleeve subsystem. Internal components that are embedded inside the intelligent sleeve are shown in dotted outline, while components visible and/or accessible from the outside world are shown in solid outlines. Components may be powered by battery power, or by induction (such as provided by magnetic coupling or inductive coupling power transfer, such as disclosed in U.S. application 2004/00000974 or WO/2005/106901 both incorporated by reference) or backscattering the RF energy from an RFID type reader as is done in passive and semi-passive RFID devices or induction (passive tags require no internal power source, (they are only active when a reader is nearby to power them), whereas semi-passive and active tags require a power source, usually a small battery) or a combination of power sources, including solar power provided from a backpack or portable device.

One common component of the memory sleeve is illustrated in FIG. 3a—memory element 3002, which is the non-volatile memory device. Memory element may be contained in an RFID transponder or tag, such as available from Maxell Corp. of America (Fair Lawn, N.J.), RFID chip model ME-Y2000 Series Maxell also supports a read/write platform for use with this chip which is contained on a printed-circuit board measuring only 50×60 mm and supports a variety of host interfaces, including USB, UART, and RS-232C standards. Other low powered memory devices, such as flash memory type devices, DRAM, low-Power DDR SDRAM, low-Power SDR SDRAM, on-chip SRAM, asynchronous and synchronous SRAM and PSRAM can be utilized.

Information may be placed in the memory sleeve at the pharmaceutical manufacturer's plant (where the vials are filled), or by communication with the host computer in an injection system (as later described). The memory should be readable by the host computer controlling the injection system. Additional information that may be stored in the memory sleeve includes, but is not limited to: (1) the date and time, provided automatically by the host computer; (2) the date code of the medication; (3) the lot number of the medication; (4) the amount of medication contained in the source vial; (5) the name or ID of the medication; (6) the manufacturer of the medication (automatically provided by the software from its internal database if it is a brand name medication rather than a generic product); (7) identification of the technician assembling the sleeves and vials; (8) a security status; and (9) a temperature status.

Various other components may be included in the memory sleeve, such as a processor, wireless communication devices (including blue tooth communication or cell phone components). Other components that may be included in the sleeve include a temperature sensor such as produced by Measurement Computing of Norton, Mass. whereby temperature logging can be used to monitor the temperature profile during transportation and storage of the vial. The temperature sensor should be positioned on the exterior of the bottle, preferably between the insulating jacket and exterior bottle surface. To conserve power, a temperature sensitive label (such as Thermax label from Thermographic Measurements Ltd in Australia) may be affixed to the vial and visual inspection of the label will indicate whether a predetermined temperature has been exceeded. Alternatively, the temperature label may be queried using a LED and light receptor to determine (based on the reflected light from the label) whether the temperature range has been violated.

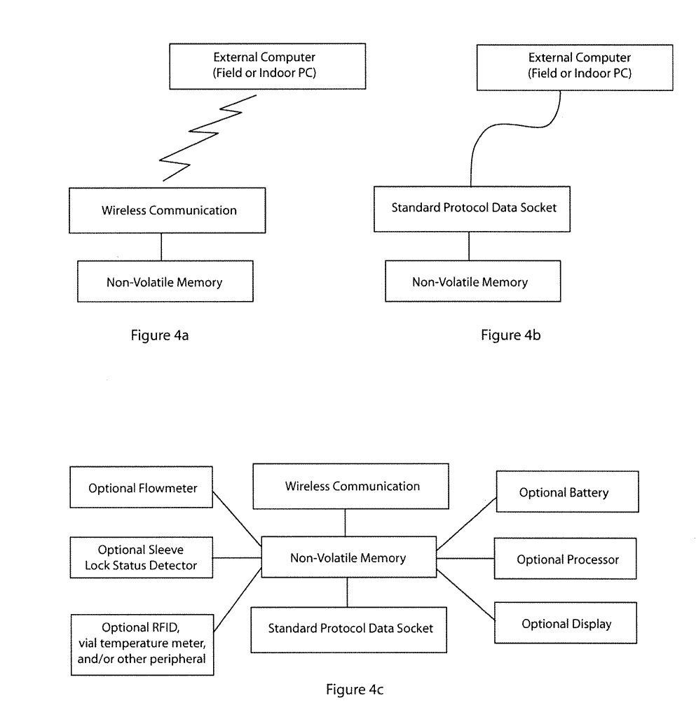

Another component that may be included is an integrity circuit that would detect if the vial seal had been tampered with. The integrity circuit can simply be a circuit detecting resistance, voltage or current across a foil hermetic seal or a conductor embedded in the seal of the vial. Other sensors can be combined with active RFID tags, including humidity, shock/vibration, light, radiation, and pressure sensors as needed. Additionally, a flow meter may be included in the sleeve to calculate fluid flows, but this is not preferred, as an ultrasonic flowmeter would be preferred (to avoid placing a meter within the vial itself) and current versions are power consumers. The memory sleeve may also include an information display screen 3003 that is capable of displaying key information about the contents/status of the vial. Connections between any of the various components are not shown for the sake of simplifying the figure. FIG. 4c shows many possible optional components that can be integrated within the memory sleeve.

FIG. 4 provides block diagram level descriptions of the memory sleeve system as it relates to its active functionality, considered in isolation from the insulating sleeve. FIG. 4a shows one base architecture, at a block diagram level, of the wireless version of the memory sleeve system. In this embodiment, communication between the external hosting computer (the application-specific software that controls the automated medication injection system) and the memory sleeve's non-volatile memory device is mediated by the wireless communication means, such as blue tooth, magnetic coupling, radio frequency communications including RFID or cell phone components (represented in the earlier FIG. 3a as element 3004). Another architecture for the memory sleeve is illustrated in FIG. 4b, where a direct connection between the external computer, or host computer, is made using one of several available industry-standard protocols, such as USB, serial connection, or other means of transmitting information in both directions via a physical cable inserted into the appropriate socket on the memory sleeve (represented in the earlier FIG. 3a as element 3001). Wireless communication is preferred as a wired interface port may become contaminated in the field. Optionally, the sleeve can incorporate both wired and wireless options (3001 and 3004 may be simultaneously present). It is understood that the physical location of such all components distributed within the sleeve shown in FIG. 3a is notional only and merely representative of a workable architecture. The chosen method to communicate with the host computer is considered a means to communicate, or a communication means.

The memory sleeve may be affixed to the vial in a number of fashions. For example, FIG. 3b illustrates a clam shell hinged annular structure for the sleeve where two half annuli are joined by an integrated hinge 3007 (the sleeve material may be sufficiently flexible to avoid a mechanical hinge) and which can be locked onto the bottle neck of the source vial using an integrated lock system or mechanical joint 3006, or by gluing, heat sealing, RF sealing or other means of fixedly joining the two ends. If the ends are connected via a locking removably attaching element, the integrity of the lock can itself be monitored by the memory sleeve. For instance, the manufacturer, after filling and attaching the sleeve, and the authorization to re-set security status, can set the status of the sleeve as “attached” or “locked.” A simple continuity circuit can be used in a clam shell embodiment, but this required periodic polling. Alternatively, the device for detecting the status of the sleeve (as attached or locked) can be a wire that is broken and must be replaced by the manufacturer to reset. This embodiment does not require polling, and can be checked upon use of the vial. If the sleeve is later removed or the lock “unlocked” this change in status can be detected and stored in memory. Re-locking or re-attaching the sleeve should not re-set the status to attached or locked unless the technician has the authority to re-set. Hence, the memory sleeve can inform a user if the sleeve/vial combination is suspect. In the hinged clam shell embodiment, it is preferred that the active elements be contained in one half of the annulus to avoid internal flexible connections through the hinge.

In a preferred embodiment, the memory sleeve is permanently attached to the vial by pharmaceutical manufacturing facility where the vial is filled and the sleeve's memory can be populated with relevant data by the manufacturer. In this embodiment, the memory sleeve is intended to be a single use device, and discarded with the vial.

### Use of the Memory Sleeve

The memory sleeve and vial can be incorporated as the reservoir bottle in an computer controlled injection system such as that disclosed in the '307 patent. In an computer controlled system, (vial with memory sleeve, pump, syringe, computer to control pump, all operatively connected) the operator would identify the vial to be used in the system to the host computer (such as by wanding the vial for RFID communications) or by establishing communications between the host computer and memory sleeve. Communications can be established by cable connections between the vial and computer (including Plug and Play type automatic recognition) or by inputting a vial ID into the host computer and having the host recognize the vial through wireless communications, or other methods (such as activating the vial (for instance, by allowing battery power to components) and having the host search for an active vial). Once communications are established between the memory sleeve and host computer, the host system can query the memory sleeve for relevant information stored in the sleeve memory (medication, integrity of vial, temperature constraints, age, etc) to insure that the proper medication is being selected and the efficacy of the medication.

Once specifics are verified, the vial can be loaded into a suitable dispensing mechanism for incorporation into the injection system (e.g. the fluid lines must be attached to the vial). Once the medication is available for dispensing, the sleeve memory, in conjunction with the host computer, can tract medication used from the vial, or medication remaining to be dispensed. This tracking can take several forms. For instance, the sleeve memory may only have the initial volume of medication stored in sleeve memory. In this event, the host computer would have to track usage against the initial volume (usage can be determined from the highly accurate system pump) to determine the amount remaining in the vial. The amount remaining (or alternatively, the amount used) can be used to determine if a new vial needs to be loaded, or, if the vial will not be exhausted in a single setting, to store volume information with a vial ID in the host computer's memory for later use of the vial.

Alternatively, if the sleeve has memory that is alterable by the host system, the host computer can send and store information on the vials fluids to the sleeve memory. For instance, after each dispensing of fluid, the host could store in sleeve memory the amount of fluid remaining in the vial (or the amount used). Note that the amount of fluid remaining in the vial must account for fluid in the lines—to account for line fluid, the system may have an initiation sequence where the lines are primed (by the pump) and the host computer tracks the volumes in the lines form the operation of the pump. Alternatively, the line volume may be a predetermined quantity, and the host computer can account for this known volume.

As described, the injection system has a host computer and a highly accurate pump, that fluidly connects a syringe with a vial and the memory sleeve that communicates with the host computer—communication may be one way or two way. A commonly employed memory in the sleeve is an RFID tag. Each RFID device is considered to have non-volatile memory, although the memory may not be alterable by the host computer. For instance, the RFID tag may only contain a vial identifier. In this instance, in conjunction with the host computer and host system database, information on the fluid remaining in the vial can be tracked by the host system if the system database contained information on the vial's initial fluid volume. Because the RFID tag's signature is tied to the initial data set entered for a given source vial, and because the RFID is also tied to all aliquots and doses extracted from the source vial, the host computer can determine the state of the source vial at any time. While the data is no longer physically associated with the source vial, but rather is associated with the source vial “virtually” in respect to a database relation, the net result produces the remaining volume.

Use with the Insulating Sleeve

In a field application, such as in a feedlot or pasture setting, the insulating sleeve is a valuable addition to the system. It should also be recognized that field deployment of many source vials can pose a considerable challenge for thermal management, whereas the passive thermal management achieved by using a insulating sleeve for each vial may keep stabilized temperatures for up to eight hours under even the most extreme outdoor temperatures.

An insulating sleeve may not be needed if the medication is not sensitive to temperature extremes, or in the event that the vials are stored in an insulated storage container or refrigerated (or heated) container and the injection system is connectable to the relevant storage device.

The vial or reservoir system allows for recognition of the “vial” (contents and status of contents for use in a controlled injection system. Recognition of the vial will reduce the possibility of operator error in administering the incorrect medication, as the host computer (or the screen on the vial or both ) can alert the operator of the error in vial ID, or that the vial is compromised (temperature, security status, or other monitored condition). Hence, the intelligent vial (vial with memory sleeve) in conjunction with the controlled injection system, allows for accurate administration of effective medications to animals, such as cattle. Further, with a disposal vial and memory sleeve, where the sleeve is permanently attached to the vial (permanent attachment means that removal would damage the functionality of the sleeve's electrical/electronic components, or removal would be detected by the sleeve) and the sleeve is programmed by the pharmaceutical manufacturer or his agent, then integrity of the medication is assured throughout the process, from manufacturing to injection.

As described, the memory sleeve is shown as a collar, but this is simply one embodiment. Other shapes are possible. The device is intended to be used in a multi-dosage environment, and typically a vial will be in the preferred range of 250 cc and larger. In the preferred embodiment, the memory sleeves is disposable, but it may be designed to be removable for a vial and re-used. In this event, the contents of the memory device would have to be reinitialized for a new use, so that the non-volatile memory device now contains information specific to the new vial of medicine with which the memory sleeve is now associated and to which it is physically attached. Further enhancements of the various embodiments of the present invention include (1) generalizing two-way communication between the memory sleeve and the generic system to be enhanced, (2) acquiring and communicating precise information about the usage of medicine being extracted from the source; and (3) the extraction and/or display of the remaining medicine and status of the medicine (e.g. temperature exceeded) within the source vial. It should be noted that the display of information could involve the actual integration of a small display screen as part of the Memory Sleeve architecture.

In an embodiment, when preparing a source vial for use with the present invention, a memory sleeve is fitted first onto the mouth and neck of the vial, and then the vial is slipped, bottom-first, into a temperature sleeve.

While a particular embodiment of the invention has been shown and described, numerous variations and alternate embodiments will occur to those skilled in the art. Accordingly, it is intended that the invention be limited only in terms of the appended claims.

1. An improved controlled injection system, comprising a host computer, a syringe, a pump, and a reservoir vial, wherein said host computer communicates with said syringe and said pump to control filling of said syringe, said vial, syringe and said pump being in fluid communication, wherein said improvement comprises a memory sleeve, said memory sleeve being fixedly attached to said vial, said memory sleeve having a memory storage device in communication with said host computer.

2. The improved controlled injection of claim 1 wherein said memory sleeve is permanently attached to said vial.

3. The improved controlled injection of claim 1 wherein said memory storage device contains data, said data comprising information that identifies a material stored in said vial.

4. The improved controlled injection of claim 3 wherein said data includes information related to a volume of material stored in said vial.

5. The improved controlled injection of claim 4 wherein said computer receives said volume information from said data, and determines the amount of material available for dispensing

6. The improved controlled injection of claim 5 wherein said computer communicates said determined amount of material available for dispensing to said memory sleeve for storage in said memory storage device.

7. A device for enhancing the accurate dispensing of injected medications in livestock, comprising:
a vial, an memory sleeve, said memory sleeve comprising a number of components, including a non-volatile memory storage device and a communications means to signal a remote device and communicate data stored in said memory storage device
said memory sleeve affixed to said vial;
said memory storage device containing information on a medication stored in said vial, said information including an identifier of said medication and a value related to the volume of material in said vial.

8. The invention of claim 7, wherein further having an insulation sleeve, said vial contained in said insulating sleeve, where said insulating sleeve is constructed from a nanoporous material.

9. The invention of claim 7, wherein said memory sleeve components further includes a processor in communication with said memory storage device and said communications means.

10. The invention of claim 7 wherein said memory sleeve comprises a collar composed of insulating material.

11. The invention of claim 7 wherein said vial includes a seal, and said components further includes a integrity circuit to detect the status of said seal.

12. The invention of claim 7 wherein said memory sleeve is removably attached to said vial and said components further induces a circuit to detect a status of the sleeve reflecting whether said sleeve has been removed from said vial.

13. The invention of claim 7, wherein the memory sleeve records at least one of the date code of the medication;
the lot number of the medication;
the amount of medication contained in the source vial;
the name of the medication;
the manufacturer of the medication; and,
identification of the technician.

14. The invention of claim 7 wherein said memory sleeve is permanently attached to said sleeve.

15. A memory sleeve for attachment to a medical vial, said memory sleeve adapted to be attached to a vial, said memory sleeve comprising a number of components, including a non-volatile memory storage device, a communications means to signal a remote device and communicate data stored in said memory storage to a remote device, and a circuit to detect the status of the sleeve as detached or attached to a vial.
